(19) United States
(12) Patent Application Publication
(10) Pub. No.: US 2005/0070853 A1
(43) Pub. Date: Mar. 31, 2005

(54) Medicament dispensing assembly

(76) Inventors: Brian Gatton, Elmer, NJ (US); John D. Buehler, Bridgeton, NJ (US); Redd Harold, Ocean View, NJ (US); Russell Granato, Vineland, NJ (US); Francisco Patterson, Mays Landing, NJ (US); David A. Manera, Petersburg, NJ (US); Michael Donegan, Franklinville, NJ (US)

Correspondence Address: Eugene E. Renz, Jr., P.C., 205 North Monroe Street, Post Office Box 2056, Media, PA, 19063-9056, US

(21) Appl. No.: 10/835,033

(22) Filed: Apr. 29, 2004

Related U.S. Application Data
(60) Provisional application No. 60/466,577, filed on Aug. 25, 2003.
(60) Provisional application No. 60/510,868, filed on Oct. 14, 2003.

(51) Int. Cl. A61M 5/00

(52) U.S. Cl. 604/187; 604/195; 604/403

(57) ABSTRACT

The combination comprising a container for a liquid medicament product having a discharge opening at one end and external screw threads, an elongated hollow barrel having a cap portion with internal threads to facilitate assembly to the container anteriorly thereof an elongated hollow plunger engageable in the barrel having an axially circumferentially extending axially directed lip of a diameter in the relaxed state slightly greater than the internal diameter of the barrel to facilitate easy movement of the plunger in the barrel to draw liquid medicament into the barrel and discharge the same on a reverse stroke, a cap of cup-like form and of an internal diameter greater than the cap portion of the barrel having a seal adapted positioned to engage the cap portion of the barrel when assembled to the container.

This application claims the benefit of Provisional Application, Ser. No. 60/466,577, entitled, ICOD ASSEMBLY, filed Apr. 30, 2003, and Provisional Application No. 60/510,868, entitled, ICOD ASSEMBLY, filed Oct. 14, 2003.

### FIELD OF THE INVENTION

The present invention relates to assemblies for dispensing liquid medicament products generally of the type shown in Buehler, et al., U.S. Pat. No. 6,273,152, entitled EXACT DOSE DISPENSING AND DEVICE ASSEMBLY, which includes a container for the medicament product and a plunger and barrel assembly for withdrawing selected quantities of medicament from the container to load the barrel which can be then disassembled from the container for expelling the product barrel.

### SUMMARY OF THE INVENTION

The present invention is an improvement over these prior assemblies and is characterized by novel features of construction and arrangement whereby the assembly can be manufactured easily and economically and wherein the parts of the assembly may be easily manipulated to selectively withdraw medicament product from the container by the user.

In the principal embodiment of the invention, the container has a stepped finish with locking means so that the barrel cap portion can be assembled over the container and seal the container in the seated position and a larger overcap can be assembled and locked to the larger stepped portion of the container which has an inner flexible seal engaging the barrel cap portion to provide a double sealing arrangement. The plunger has an axially directed sealing lip which is of a diameter in the relaxed state slightly greater than the internal bore diameter of the barrel to facilitate ease of movement of the plunger in the barrel.

In use therefore, the overcap is simply removed by pressing it downwardly and rotating it relative to the locking elements of the container to a release position. With the overcap removed, the finger grip portion of the plunger can now be withdrawn axially to draw a predetermined quantity of the liquid medicament into the barrel. When the desired quantity has been drawn into the barrel, the cap portion is rotated so that the plunger and barrel are now free and the medicament can be dispensed.

In accordance with another embodiment, the barrel is connected to concentric depending spaced flanges which engage in the container opening to define a generally cylindrical cavity and the plunger has a locking hook which engages with a locking lug on the wall of the overcap. The outer end of the plunger and flexible lip create a seal and an upward bias to lock the plunger inside the barrel. When the user desires to activate the syringe, the plunger is pushed axially downwardly against the bias of the flexible lip and rotated to release it from the locking lug permitting axial withdrawal of the plunger to draw liquid medicaments into the barrel. The overcap can then be activated to release it from the container and permit discharge of the contents drawn into the barrel. This locking feature on the plunger obviates the need for an overcap

### DESCRIPTION OF THE DRAWINGS

These and other objects of the present invention and the features and details thereof are hereinafter more fully set forth with reference to the accompanying drawings wherein:

FIG. 1. is an exploded side elevational view of the components comprising the medicament dispensing assembly of the present invention, including the helical lock overcap, plunger, cap barrel combination and bottle or container;

FIG. 2. is a side elevational view of the assembly shown in FIG. 1 in the assembled relation;

FIG. 3. is an exploded transverse sectional view of the components comprising the assembly shown in FIG. 1;

FIG. 4 is an enlarged sectional view taken on lines 4-4 of FIG. 2 showing the parts of the assembly in the assembled condition;

FIG. 5a is a side elevational view of the medicament dispensing assembly with the overcap shown in section so the internal parts can be seen more clearly;

FIG. 5b is an enlarged sectional view of the portion circled and identify in 5a and 5b;

FIG. 5c is a side elevational view similar to FIG. 5a with showing the overcap being removed;

FIG. 5d shows the lug position relative to the ramps during removal and is the circled portion identified in 5c and 5d;

FIG. 6a is a transverse sectional view showing the plunger being activated relative to the barrel to remove liquid product from the container;

FIGS. 6b and 6c are enlarged sectional views of the portions encircled and identified as 6b and 6c in FIG. 6a;

FIG. 7a shows the plunger and barrel removed from the container;

FIG. 7b shows the plunger activated forwardly in the barrel to dispense product;

FIG. 8 is a transverse sectional view of another liquid medicament dispensing assembly in accordance with the present invention;

FIG. 9 is a view similar to FIG. 8 showing the plunger in cross section;

FIGS. 9a and 9b are enlarged sectional views of the portioned circled 9a and 9b in FIG. 9;

FIG. 10 is a view of the parts before activation of the plunger; and

FIG. 11 is a view similar to FIG. 10 showing the plunger in a retracted position to draw medicament contents into the barrel.

FIG. 12 is an exploded side elevational view of another embodiment of medicament dispensing assembly in accordance with the present invention;

FIG. 13 is a side elevational view of the parts in the assembled relation;

FIG. 14 is a top plan view of the outer cap;

FIG. 15 is a transverse sectional view through the outer cap taken on lines 15-15 of FIG. 14;

FIG. 16 is a bottom plan view of the outer cap;

FIG. 17 is a top plan view of the inner cap and barrel assembly;

FIG. 18 is a side elevational view of the inner cap barrel assembly;

FIG. 19 is a sectional view taken on lines 19-19 of FIG. 17;

FIG. 20 is a side elevational view partly in section showing the inner cap nested in the outer cap;

FIG. 21 is a view similar to FIG. 20 showing the outer cap in a fully depressed position so the inner engaging cam elements engage permitting rotation of the cap and a direction to remove the inner cap from the container;

FIG. 22 is a view similar to FIG. 21 showing the cam elements on the inner and outer cap center engaged to permit rotation into the cap in the direction indicated by the arrows;

FIG. 22a is a fragmentary view of the portion encircled in FIG. 22;

FIG. 23 is a transverse sectional view showing the parts of the dispensing assembly in the fully housed position;

FIG. 24 is a similar sectional view showing the plunger moving to its outer limit position to draw a quantity of medicament product into the plunger; and

FIG. 24a is an enlarged fragmentary view of the portion encircled in FIG. 24.

### DESCRIPTION OF THE PREFERRED EMBODIMENTS

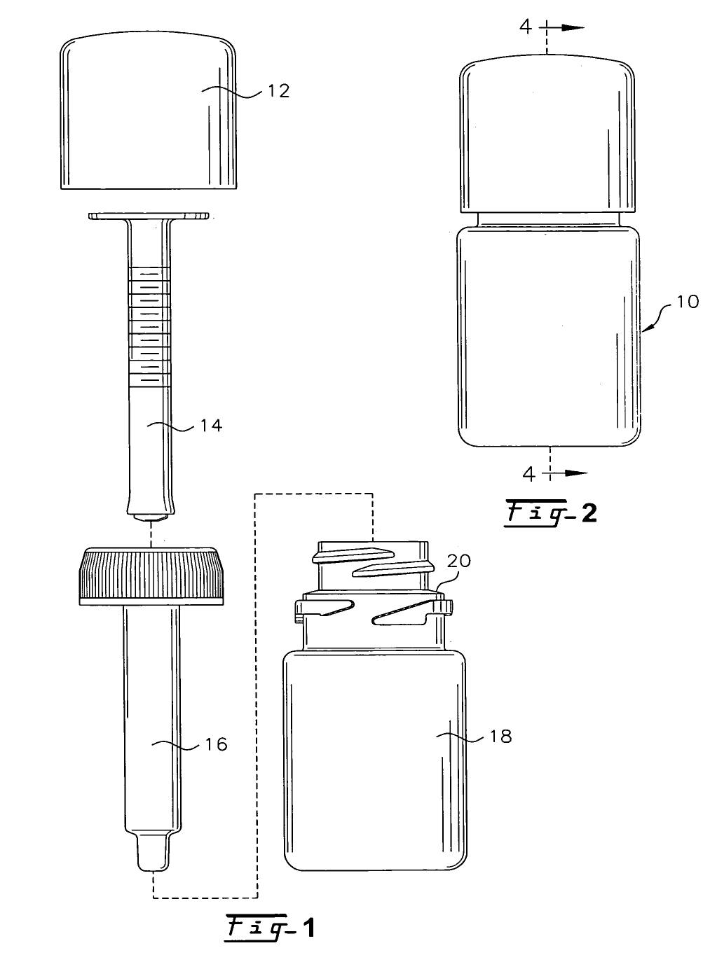

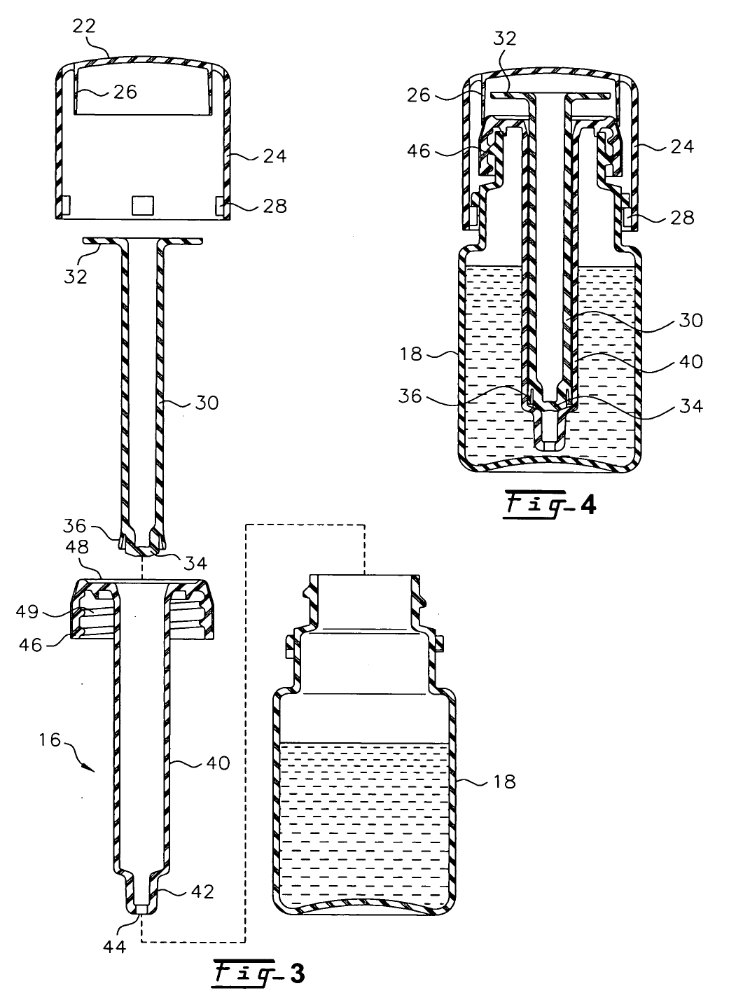

Referring now to the drawings and particularly to FIGS. 1-7 inclusive, there is shown a combination cap and container assembly for dispensing liquid medicaments generally referenced by the numeral (10). The assembly comprises an overcap (12) of inverted cup-like form, an elongated tubular plunger (14), a barrel (16) and a container (18) having a container finish (20).

Considering now the components in more detail, the overcap (12) comprises a top (22) and a circumferentially extending skirt (24) depending therefrom. A circumferentially extending flange (26) depends from the interior wall of the top (22) and is spaced radially inwardly from the skirt or sidewall (24). The overcap (22) has a series of circumferentially spaced radially inwardly directed lugs (28) adjacent its lower open end.

The plunger has an elongated body portion (30) and an outwardly directed circumferentially extending flange (32) at its upper end. The plunger has a radially outwardly, circumferentially extending sealing lip (36) adjacent its lower end which is of a diameter to snugly engage the interior wall of the barrel as shown in FIG. 4.

The barrel (16) is also an elongated hollow body portion (40) which is stepped down to define a nose (42) at its outer end and has a discharge opening (44). The barrel has a cap section (46) formed integrally therewith and connected to the main portion of the barrel by a radial wall (48). The cap portion has internal spiral threads (49) to mate with threads (20) on the container (18) to secure the barrel (16) in place as explained in more detail hereafter. The connecting wall (48) has a circumferentially extending sealing lip (50) depending therefrom to function as a seal as shown in FIG. 4.

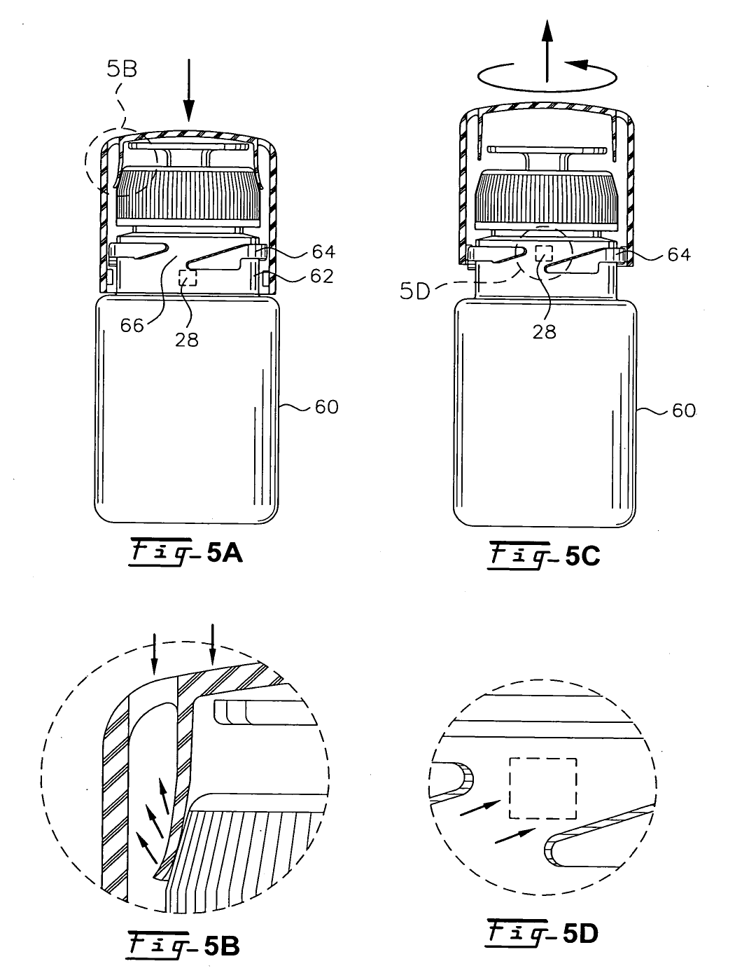

The container (18) as illustrated has a generally cylindrical shape comprising an enlarged body portion (60), a reduced first neck portion (62) having a series of circumferentially extending locking elements (64) spaced apart to define an angle or ramp (66) so that when the overcap (12) is turned to seat it in place, the lugs (28) pass through the space S between the locking elements (64) in the manner shown in FIGS. 5c and 5d to lock the parts in place. When the parts are fully assembled, the sealing lip (26) engages and seals with the outwardly tapered circumferential wall of the cap portion (46) of the barrel in the manner shown in FIG. 5a.

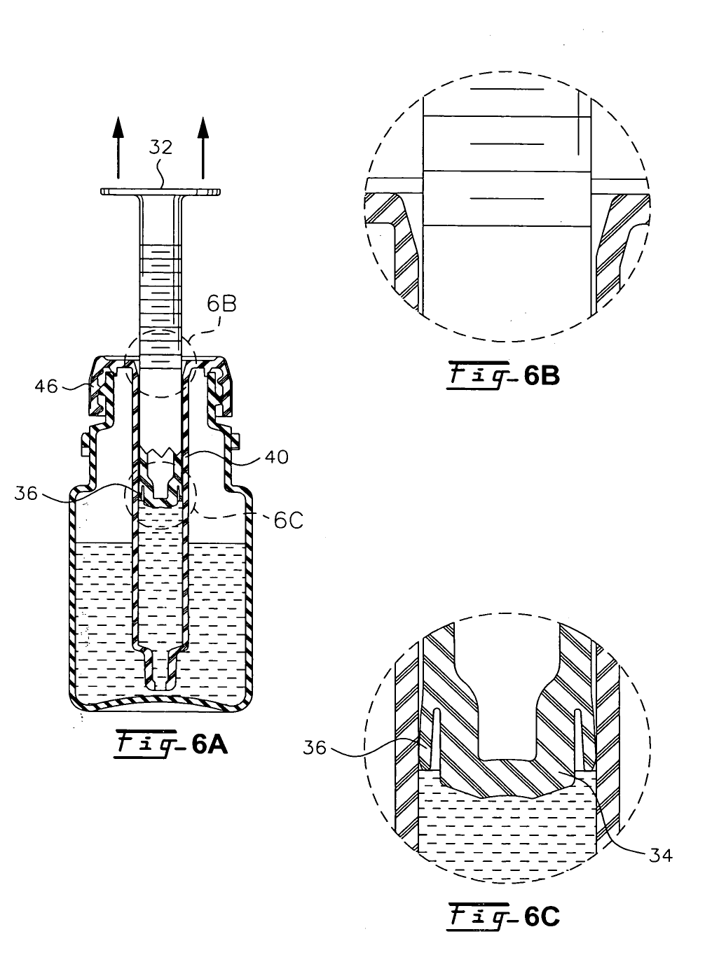

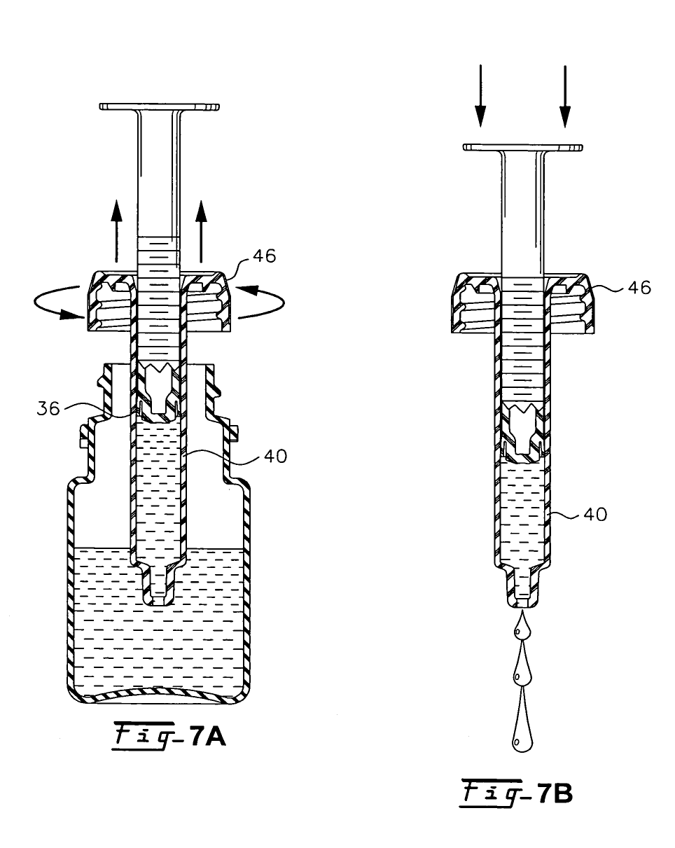

In using the assembly, the overcap (12) is removed so that the plunger (14) can be withdrawn axially relative to the barrel (16) to draw a quantity of the medicament into the barrel in the manner shown in FIG. 6a. When the desired quantity has been drawn into the barrel, the cap portion (46) is rotated in the direction to release it from the container so that the plunger and barrel are now free and the product can be dispensed in the manner shown in FIG. 7b.

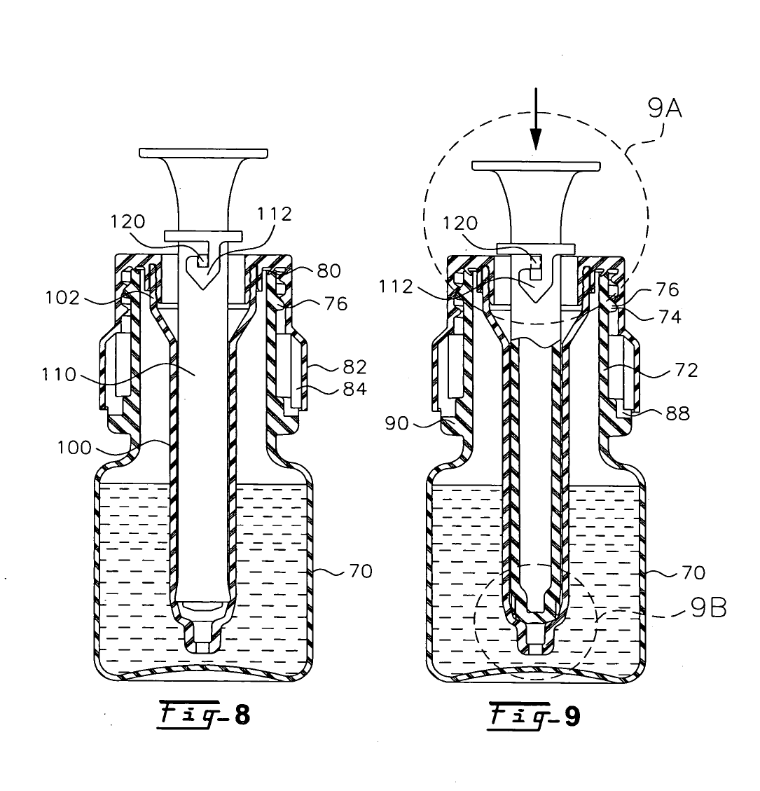

There is shown in FIGS. 8-11 inclusive another embodiment of dispensing assembly in accordance with the present invention. This assembly comprises a container (70) for a quantity of liquid medicament to be dispensed having an elongated reduced neck portion (72) which has spiral screw threads (74) formed in the outer periphery. A cap (80) fits over the screw threads (74) and mates therewith with complementary threads (76). The lower skirt portion (82) is offset and enlarged. A pair of internal fins (84) which are diametrically spaced engage in notched recesses (88) formed in a radially outwardly directed bead or projection (90) on the bottle neck finish (72). The barrel (100) has an enlarged upper portion as at (102) which snugly engages in a pair of spaced flanges (103, 105) on the inside of the cap (80). The plunger (110) engages in the barrel and has a hook (112) formed at its upper end which engages a locking lug (120) on the interior of the cap.

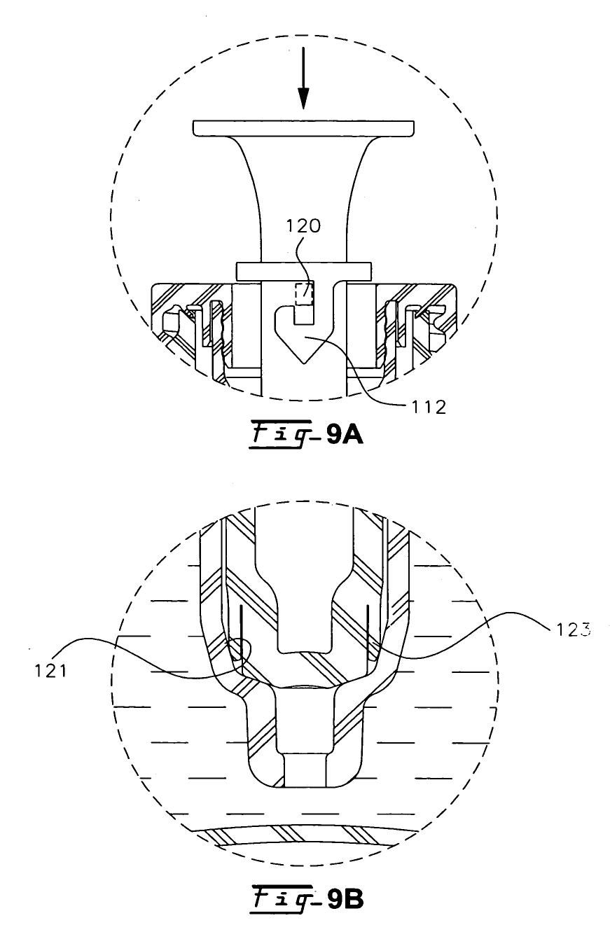

When it is desired to activate the system, the plunger is pushed downwardly and rotated until the hook (112) is displaced angularly and no longer aligned with the locking lug (120) to permit the plunger to be withdrawn axially to draw a quantity of the liquid medicament into the barrel in the manner shown in FIG. 11. Note that the bore of the barrel (100) is tapered inwardly and downwardly as at 121 at an angle of about 14 degrees to the axis of the barrel as shown in FIG. 9b the sealing lip (123) are compressed somewhat when the plunger (110) is pressed downwardly to release from the locking lock (120) and provides a spring action to lock the parts when the plunger is released and the hook (112) is aligned with the lug (120). The skirt portion of the cap (80) is squeezed radially inwardly in the region between the locking ribs (84) to expand the skirt sufficiently to displace the fins (84) radially outwardly to permit rotation of the cap for removal.

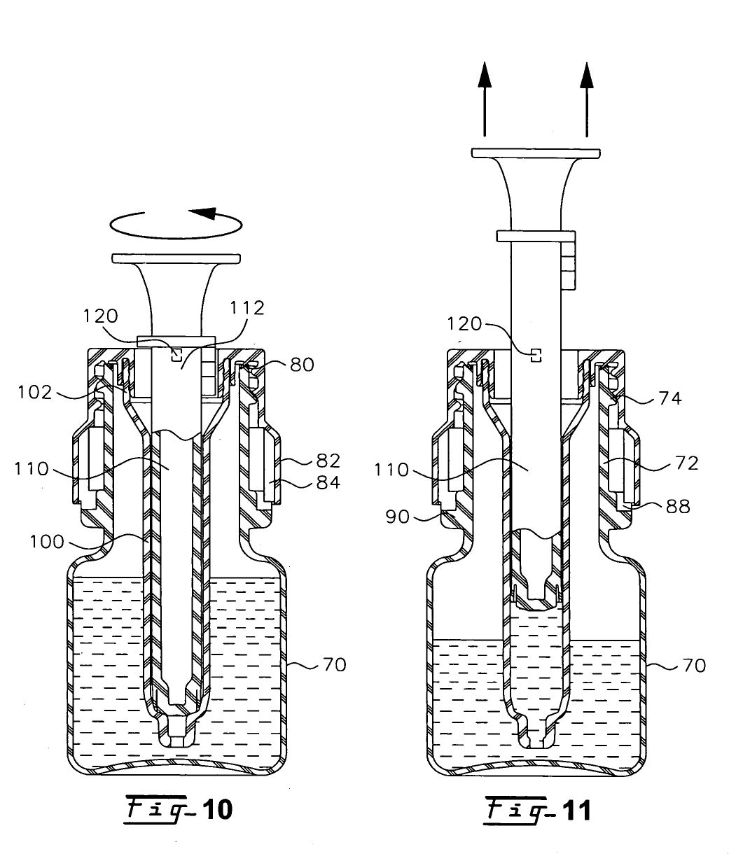

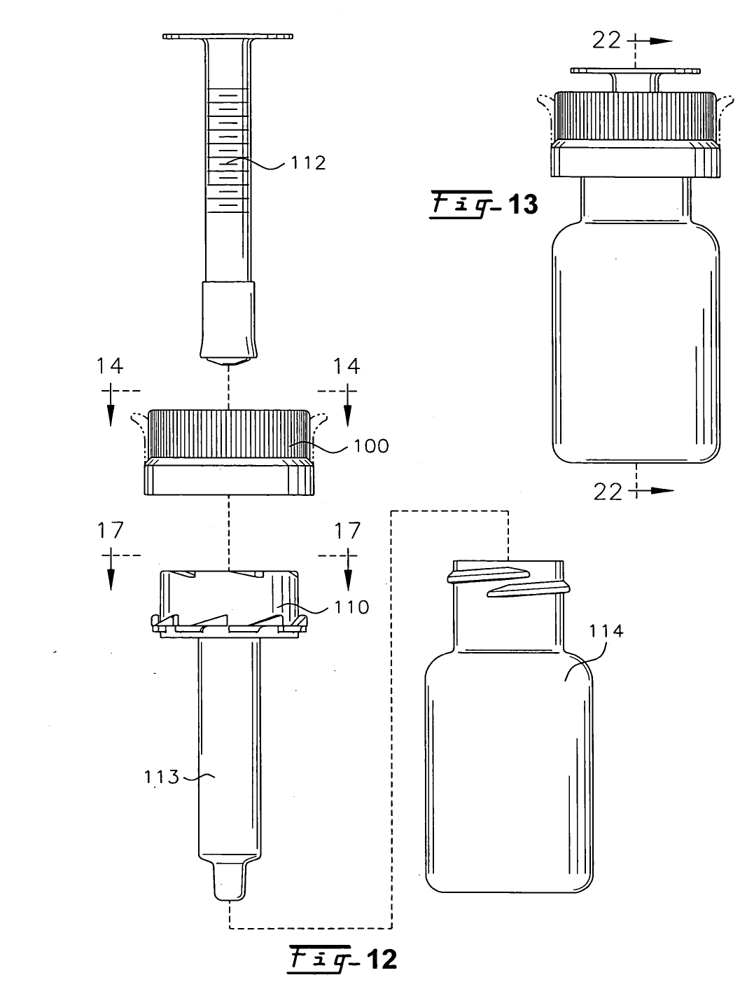

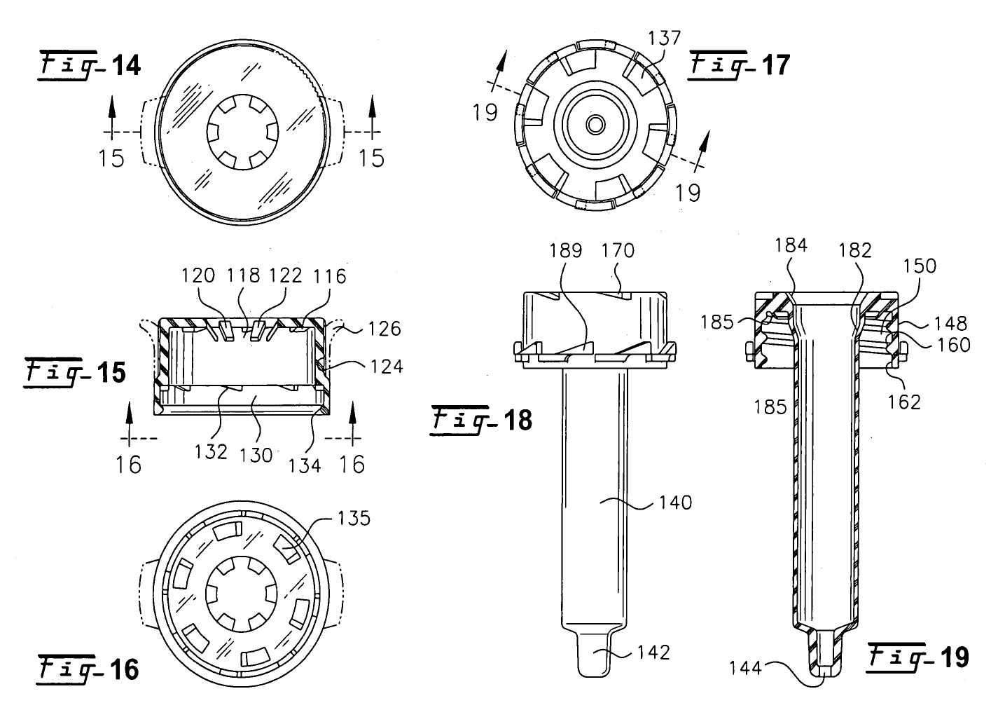

There is shown in FIGS. 12-24 inclusive another embodiment of medicament dispensing assembly in accordance with the present invention. The assembly as best illustrated in FIGS. 12 and 13 comprises an outer cap (100), an inner cap assembly (110), an elongated plunger (112) adapted to engage in the barrel (113) through an opening (115) in the outer cap of the container (114).

The outer cap (100) which is generally of cup-like form has an annular top (116) defining a central opening (118) which mounts a series of circumferentially spaced downwardly angled flexible spring fingers (122). The cap has a circumferentially extending skirt (124) extending downwardly from the outer peripheral edge of the annular portion (116) which has formed therein a pair of gently outwardly curved finger-grip portions (126) at diametrically opposed locations. The skirt (124) is stepped and offset outwardly at its lower portion to define a juncture (130) which has a series of circumferentially spaced cams (132) mounted thereon which depend downwardly in an axial direction. The outer edge of the skirt (124) has an inwardly directed bead (134) which snap fits over the inner cap in a manner described hereafter. The outer cap (100) also has a series of radially directed protrusions (135) which are circumferentially spaced and selectively engage in complementary pockets (137) in the inner cap (110) to permit activation of the inner cap and turn it in a direction either to remove it from the container or apply it to the container. This configuration is shown in some of the embodiments illustrated in U.S. Pat. No. 6,273,152 incorporated herein by reference.

The inner cap assembly comprises an elongated generally cylindrical hollow barrel (140) stepped to define a tip portion (142) having an opening (144) therein. The cap portion (146) which is formed integrally with the inner end of the barrel (140) has a generally circumferentially extending skirt (148) within internal screw threads (150) which mate with the screw threads on the finish of the container to permit the plunger to be assembled and removed in a manner described hereafter. The skirt has a series of inclined ramps (160) which are circumferentially spaced on the outer peripheral surface. The connecting wall (162) for connecting the skirt to the barrel has a series of circumferentially spaced cavities or cutouts (170). The upper end of the barrel is bulged out slightly as at (182) and the inner edge of the connecting wall is beveled outwardly as at (184) for purpose to be described hereafter.

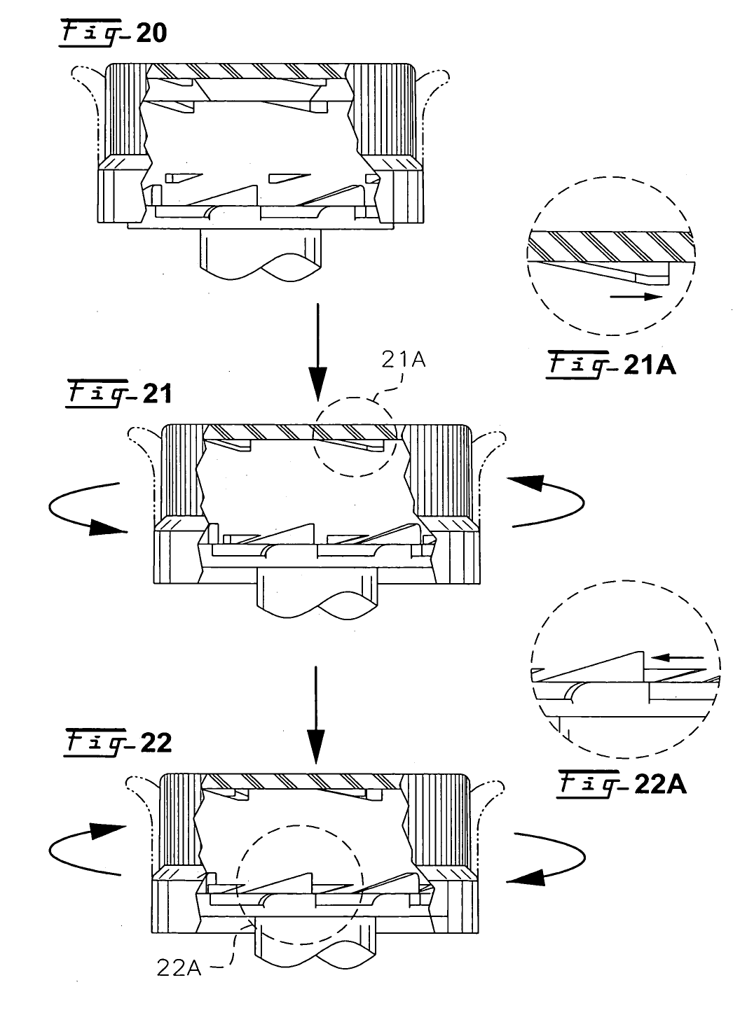

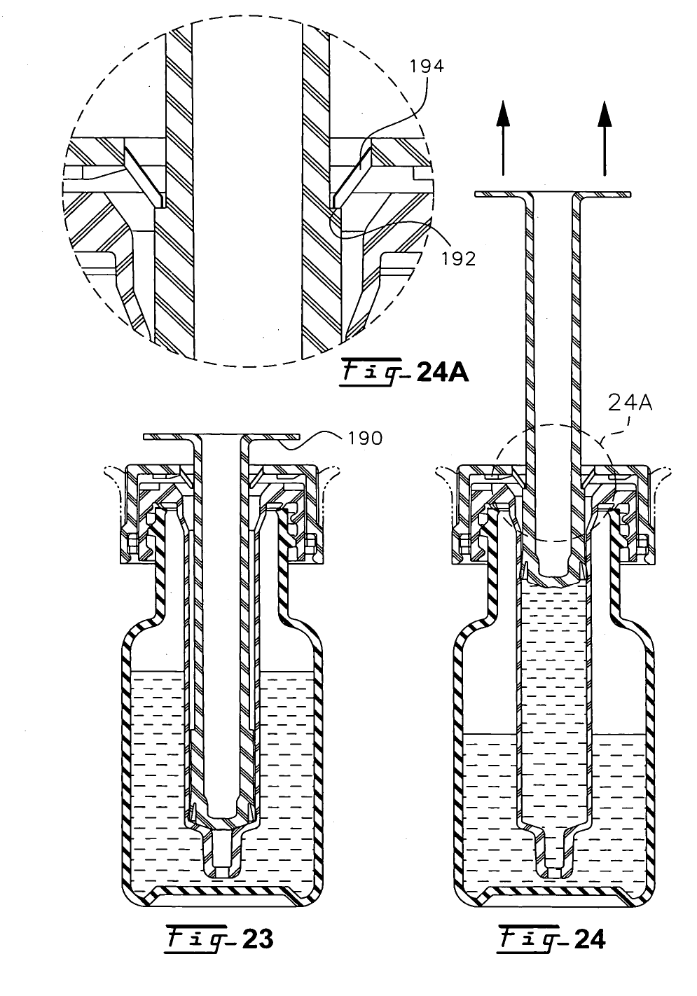

Considering now an operation of the dispensing assembly described above. The parts comprising the assembly are initially in the position shown in FIG. 22 wherein the inner cap is fully threaded inwardly and seated on the container neck in the manner shown and wherein the seal (185) of the inner cap (110) engages the outer axial end face of the container finish in the manner shown in FIG. 22. The inner and outer caps are generally similar to the inner and outer caps of the dispensing device shown in U.S. Pat. No. 6,273,152. Thus the cam configuration on the inner and outer caps is such that when the outer cap is rotated in one direction, the cams (187, 189) simply override where as rotation of the outer cap in the opposite direction causes interengagement of the cams (187, 189) and rotation of the inner cap in a direction to thread it off of the container when it is desired to discharge medicament products from the barrel (140). Referring now to FIGS. 22-24 inclusive, when it is desired to draw a predetermined quantity of medicament from the container into the barrel, the plunger is simply withdrawn in an axial direction by engaging the flange (190) at its upper end and pulling it outwardly in the manner shown in FIG. 23. It is noted that the plunger has a stepped cross-sectional configuration defining a circumferentially extending abutment or ledge (192) which is engaged by the fingers (194) which project downwardly from the outer cap in the manner shown in FIG. 24 to limit the displacement of the plunger in the barrel in the manner shown.

Even though particular embodiments of the present invention have been illustrated and described herein, it is not intended to limit the invention and changes and modifications may be made therein within the scope of the following claim.

1. The combination comprising a container for a liquid medicament product having a discharge opening at one end and external screw threads, an elongated hollow barrel having a cap portion with internal threads to facilitate assembly to the container anteriorly thereof an elongated hollow plunger engageable in the barrel having an axially circumferentially extending axially directed lip of a diameter in the relaxed state slightly greater than the internal diameter of the barrel to facilitate easy movement of the plunger in the barrel to draw liquid medicament into the barrel and discharge the same on a reverse stroke, a cap of cup-like form and of an internal diameter greater than the cap portion of the barrel having a seal adapted positioned to engage the cap portion of the barrel when assembled to the container.

2. The combination as claimed in claim 1 wherein said cap portion has a circumferentially extending depending sealing lip which engages interiorly the container discharge opening when the barrel and cap portion are fully seated on the container.

3. The combination as claimed in claim 1 wherein said cap has a circumferentially extending depending skirt having inwardly projecting locking lugs which cooperate with locking means on the container finish for selectively applying and removing the cap from the container and including a circumferentially extending sealing flange depending from the inside of the top of the cap and spaced radially inwardly from the skirt which engages the cap portion of the barrel when the cap is seated on the container in a locked position.

4. The combination comprising a container for a liquid medicament product having a discharge opening at one end, an elongated hollow barrel, an elongated hollow plunger engageable in the barrel, a cap of cup-like form means for securing the barrel at its upper end to the cap and means for locking the plunger in the barrel in a fully assembled state.

5. The combination as claimed in claim 4 wherein the barrel terminates in an offset enlarged upper portion which engages between a pair of circumferentially extending radially spaced flanges depending from the inside top of the cap and wherein the confronting faces of the locking flanges have a series of interengaging locking fingers.
